(19) United States
(12) Patent Application Publication
(10) Pub. No.: US 2026/0013823 A1
(43) Pub. Date: Jan. 15, 2026

(54) METHODS AND SYSTEMS FOR CENTERING AN X-RAY IMAGING SYSTEM

(71) Applicant: Siemens Healthineers AG, Forchheim (DE)

(72) Inventor: Magdalena HERBST, Erlangen (DE)

(73) Assignee: Siemens Healthineers AG, Forchheim (DE)

(21) Appl. No.: 19/260,689

(22) Filed: Jul. 7, 2025

(30) Foreign Application Priority Data
Jul. 9, 2024 (DE) 10 2024 206 450.5

(51) Int. Cl. A61B 6/58; A61B 6/00; A61B 6/06; G01T 7/00

(52) U.S. Cl. CPC A61B 6/587; A61B 6/06; A61B 6/54; A61B 6/582; G01T 7/00

(57) ABSTRACT

A method for centering an X-ray imaging system with an emitter with a variable pose and an X-ray detector with a variable pose which can be controlled independently of the variable pose of the emitter involves controlling a predetermined pose with the emitter and controlling a pose of the X-ray detector corresponding to the predetermined pose of the emitter. Subsequently, the actual pose of the emitter and the actual pose of the X-ray detector is determined. Furthermore, a first deviation of the actual pose of the emitter from the predetermined pose of the emitter is determined and a second deviation of the actual pose of the X-ray detector from the predetermined pose of the X-ray detector is determined. Finally, a corrected predetermined pose for centering the emitter based on the first deviation and a corrected predetermined pose for centering the X-ray detector based on the second deviation is determined.

### CROSS-REFERENCE TO RELATED APPLICATION(S)

The present application claims priority under 35 U.S.C. § 119 to German Patent Application No. 10 2024 206 450.5, filed Jul. 9, 2024, the entire contents of which are incorporated herein by reference.

### FIELD

One or more example embodiments relates to a method for centering an X-ray imaging system with an emitter with a variable pose and an X-ray detector with a variable pose which can be controlled independently of the variable pose of the emitter. One or more example embodiments also relates to a centering facility (also referred to as a centering device). Furthermore, one or more example embodiments relates to an X-ray imaging system.

### RELATED ART

With flexible robotic X-ray imaging systems, such as for example C-arms or robot-supported, ceiling-guided X-ray imaging systems, in addition to 2D images, 3D images (cone-beam CT, CBCT) can also be produced. Above all if the X-ray tube and the X-ray detector are not mechanically coupled, as is the case with the C-arm, very precise mechanical adjustment of the system must be carried out so that the position and orientation of the X-ray tube and the X-ray detector are optimal in relation to one another in all spatial positions. This requires a lot of time and experienced service technicians. In addition, parts of the system may become deformed during movement, which also leads to deviations. Typically, the telescopic arms to which the X-ray tube and the X-ray detector are attached bend slightly due to centrifugal forces; the more they are extended, the more they bend.

The actual positions of the system are known through geometry calibration and therefore the reconstruction can be carried out correctly. Nevertheless, there is the problem of the central beam not always striking the center of the detector. This has the following consequence for the image:
The position of the collimator on the projection cannot be correctly determined as it is assumed that the center of the detector is struck.
The resulting image field is limited as the existing detector surface is not used. In addition, an area which is not covered by the detector and cannot be used is irradiated.
If the collimator edges are located in the reconstructed area, this creates a bright edge at the edge of the image field.

### SUMMARY

Until now, the projections were cropped an additional 2 cm on each side for real 3D images to ensure that the collimator edge did not create any artifacts in the reconstructed volume. If the deviations were very large, the system adjustment was repeated until the desired result was achieved. In this case, therefore, more effort was required to repeat the system adjustment.

The inventors have discovered a method and an apparatus for adjusting an X-ray imaging system with which the aforementioned problems are at least mitigated.

This is achieved by a method for centering an X-ray imaging system with an emitter with a variable pose and an X-ray detector with a variable pose which can be controlled independently of the variable pose of the emitter as claimed in claim 1, a centering facility as claimed in claim 11 and an X-ray imaging system as claimed in claim 13.

### BRIEF DESCRIPTION OF THE DRAWINGS

Example embodiments explained in more detail hereinafter with reference to the attached figures on the basis of exemplary embodiments. The figures show:

FIG. 1 illustrates a diagrammatic view of a phantom with a number of opaque spheres which are arranged in a spiral,

FIG. 2 illustrates a diagrammatic top view of the trajectory of an emitter and an X-ray detector of an X-ray imaging system,

FIG. 3 illustrates a top view of an X-ray imaging system with an emitter and an X-ray detector and the trajectories already shown in FIG. 2 as well as the beam path of the X-rays emitted by the emitter,

FIG. 4 illustrates a top view of the X-ray imaging system already shown in FIG. 3, the trajectory of the X-ray detector being slightly shifted and twisted compared to an ideal trajectory,

FIG. 5 illustrates a top view of the X-ray imaging system already shown in FIG. 3 and FIG. 4, the trajectory of the X-ray detector having been corrected,

FIG. 6 illustrates a flow chart which illustrates the method for centering an X-ray imaging system with an emitter with a variable pose and an X-ray detector with a variable pose according to an exemplary embodiment of the invention,

FIG. 7 illustrates a diagrammatic view of a centering facility according to an exemplary embodiment of the invention, and

FIG. 8 illustrates a diagrammatic view of an X-ray imaging system according to an exemplary embodiment of the invention.

### DETAILED DESCRIPTION

In the method according to one or more example embodiments for centering an X-ray imaging system with an emitter with a variable pose and an X-ray detector with a variable pose which can be controlled independently of the variable pose of the emitter, a predetermined pose Pi_0_S is first controlled with the emitter. In this context, “centering” should be understood to mean that the emitter and the X-ray detector are aligned with one another in such a way that the X-rays generated by the emitter are centered on the X-ray detector. “Pose” should be understood as the position combined with the orientation of an object. “Variable pose” should be understood here to mean that the pose of the emitter and the X-ray detector can be flexibly fixed via control and that the emitter and the X-ray detector are not permanently mounted on a possibly rotatable mounting bracket. In particular, the X-ray tube and the X-ray detector, which can be controlled independently of one another, are not mechanically coupled to one another. It should therefore be possible to change the poses of the emitter and the X-ray detector in particular relative to one another. In contrast to a usual fixed mounting of an emitter and an X-ray detector, for example on a drum, in the case of a mechanical coupling, the two elements of the X-ray imaging system can in principle therefore be positioned and oriented independently of one another with regard to their pose and can therefore be freely controlled. The “predetermined pose” represents a target pose which is specified by an imaging protocol. However, this target pose can differ slightly from the actual pose, i.e. the actual pose of the emitter or the X-ray detector, due to the aforementioned flexibility of the system.

Furthermore, a pose Pi_0_D of the X-ray detector corresponding to the predetermined pose Pi_0_S of the emitter is controlled. In this context, “corresponding” should be understood to mean that the X-ray detector is positioned exactly opposite the emitter in such a way that the orientation of the detector surface matches the beam direction and the beam center of the X-ray beam exactly intersects the center of the detector surface of the X-ray detector.

After control, in a next step, a calibration step, the actual pose Pa_0_S of the emitter is determined by measuring this pose, which was achieved as a result of the control. Likewise, the actual pose Pa_0_D of the X-ray detector is determined. These actual poses or calibrated poses may deviate from the aforementioned target poses Pi_0_S, Pi_0_D, in particular as the emitter and the X-ray detector can be moved mechanically independently of one another.

Based on the measured values determined in this way, a first deviation AS of the actual pose Pa_0_S of the emitter is then determined from the predetermined pose Pi_0_S of the emitter:

$$AS = Pa\_ 0 \_S - Pi\_ 0 \_S . \qquad ( 1 )$$

Likewise, a second deviation of the actual pose Pa_0_D of the X-ray detector is determined from the predetermined pose Pi_0_D of the X-ray detector based on the measured values:

$$AD = Pa\_ 0 \_D - Pi\_ 0 \_D . \qquad ( 2 )$$

Such a deviation AS, AD can, for example, be determined by a vector of a difference between the vector of the original predetermined pose and the vector of the actual pose of a component. The position and orientation can also be considered and evaluated separately, standards of the difference between position vectors and orientation vectors then being considered.

Based on the first deviation AS, a corrected predetermined pose Pi_1_S for centering the emitter is determined in a step for obtaining adjustment data:

$$Pi\_ 1 \_S = Pi\_ 0 \_S - AS . \qquad ( 3 )$$

Based on the second deviation AD, a corrected predetermined pose Pi_1_D for centering the X-ray detector is determined:

$$Pi\_ 1 \_D = Pi\_ 0 \_D - AD . \qquad ( 4 )$$

The target pose Pi_0_S, Pi_0_D of the respective component is therefore modified in such a way that the respective deviation determined via measurement is reduced or disappears completely in the ideal case.

Finally, preferably in an adjustment step, the emitter is controlled with the determined corrected predetermined pose Pi_1_S of the emitter and the X-ray detector is controlled with the determined corrected predetermined pose Pi_1_D of the X-ray detector, as a result of which centering is realized.

As explained in detail later, the method according to one or more example embodiments can also be repeated or even carried out iteratively several times, the deviation AS_n, AD_n of the actual pose Pa_n_S, Pa_n_D

$$AS\_n = Pa\_n \_S - Pi\_ 0 \_S , \qquad ( 5 )$$

$$AD\_n = Pa\_n \_D - Pi\_ 0 \_D , \qquad ( 6 )$$

from the original predetermined pose (n=1, 2, 3, etc.) becoming smaller and smaller until it falls below a predetermined threshold value ASW:

Centering preferably takes place in such a way that a center beam of the emitter strikes the center of the X-ray detector and the center beam strikes the detector surface of the X-ray detector vertically.

The actual poses of the emitter and the X-ray detector can also be determined simultaneously, above all when a calibration takes place with a spiral phantom.

Advantageously, a pose of an emitter and an X-ray detector of an X-ray imaging system can be precisely controlled by the calibration and adjustment process according to one or more example embodiments. In this manner, in particular the occurrence of a disturbing image of a collimator on the imaging surface of the X-ray detector is avoided. In general, the imaging surface of the X-ray detector can be fully utilized for imaging. In particular, it is possible to avoid an imaging procedure having to be repeated because the emitter and the collimator were not precisely aligned with one another.

The centering facility according to one or more example embodiments has a control unit for controlling a predetermined pose Pi_0_S with the emitter and for controlling a corresponding pose Pi_0_D of the X-ray detector which can be controlled independently of the predetermined pose Pi_0_S of the emitter.

The centering facility according to one or more example embodiments also comprises a measuring unit for determining the actual pose Pa_0_S of the emitter and for determining the actual pose Pa_0_D of the X-ray detector.

In addition, the centering facility according to one or more example embodiments has a deviation determination unit for determining a first deviation AS of the actual pose Pa_0_S of the emitter from the predetermined pose Pi_0_S of the emitter and for determining a second deviation AD of the actual pose Pa_0_D of the X-ray detector from the predetermined pose Pi_0_D of the X-ray detector.

Finally, the centering facility according to one or more example embodiments comprises a correction determination unit for determining a corrected predetermined pose Pi_1_S for centering the emitter based on the first deviation AS and for determining a corrected predetermined pose Pi_1_D for centering the X-ray detector based on the second deviation AD. The centering facility according to one or more example embodiments shares the advantages of the method according to one or more example embodiments for centering an X-ray imaging system with an emitter with a variable pose and an X-ray detector with a variable pose which can be controlled independently of the variable pose of the emitter.

The control unit of the centering facility according to one or more example embodiments is preferably configured to control the corrected predetermined pose Pi_1_S of the emitter and the corrected predetermined pose Pi_1_D of the X-ray detector. Advantageously, not only a correction of the target poses is carried out, but this correction can also be implemented by a control so that the poses of the emitter and the X-ray detector are adjusted or centered.

The X-ray imaging system according to one or more example embodiments has an emitter with a variable pose control, an X-ray detector with a pose which can be variably controlled independently of the variable pose of the emitter, and a centering facility according to one or more example embodiments. The X-ray imaging system shares the advantages of the centering facility according to one or more example embodiments and the method according to one or more example embodiments for centering an X-ray imaging system with an emitter with a variable pose and an X-ray detector with a variable pose which can be controlled independently of the variable pose of the emitter.

The majority of the aforementioned components of the centering facility according to one or more example embodiments can be implemented in whole or in part in the form of software modules in a processor of a corresponding computer system, for example by a control facility (also referred to as a controller) of an X-ray imaging system or a computer which is used to control such a system. The advantage of a largely software-based realization is that previously used computer systems can also be retrofitted with ease via a software update in order to work in the manner according to one or more example embodiments.

In this respect, the object is also achieved by a corresponding computer program product with a computer program which can be loaded directly into a computer system, with program sections to carry out the steps of the method according to one or more example embodiments for centering an X-ray imaging system with an emitter with a variable pose and an X-ray detector with a variable pose which can be controlled independently of the variable pose of the emitter, when the program is executed in the computer system. In addition to the computer program, such a computer program product may possibly include additional elements such as, for example, documentation and/or additional components, including hardware components such as, for example, hardware keys (dongles, etc.) for using the software.

A computer-readable medium, for example a memory stick, a hard disk or another transportable or permanently installed data carrier, on which the program sections of the computer program which can be read in and executed by a computer system are stored, can be used for transport to the computer system or to the control facility and/or for storage on or in the computer system or the control facility. The computer system can, for example, have one or more cooperating microprocessors or the like for this purpose.

The dependent claims and the following description each contain particularly advantageous embodiments and developments of one or more example embodiments. In particular, the claims of one claim category can also be developed analogously to the dependent claims of another claim category. In addition, within the scope of one or more example embodiments, the various features of different exemplary embodiments and claims can also be combined to form new exemplary embodiments.

In a preferred variant of the method according to one or more example embodiments for centering an X-ray imaging system with an emitter with a variable pose and an X-ray detector with a variable pose which can be controlled independently of the variable pose of the emitter, the steps of the method are repeated in the event that the first deviation AS and/or the second deviation AD exceed a predetermined threshold value ASW, the corrected predetermined poses Pi_1_S, Pi_1_D being used as predetermined poses during the repetition. The corrected predetermined poses Pi_1_S, Pi_1_D are used for control during the repetition, but the values of the original predetermined poses Pi_0_S, Pi_0_D are also used for the determination of the first and second deviation during the repetition. This is necessary because the original target values serve as a comparison with the current actual values when determining the deviations. Advantageously, the threshold value ASW is used as a quality criterion for the accuracy of the centering, the requisite accuracy possibly being achieved via repetition of the method.

In an equally preferred variant of the method according to one or more example embodiments for centering an X-ray imaging system with an emitter with a variable pose and an X-ray detector with a variable pose, the steps of the method are repeated iteratively until the first deviation AS and/or the second deviation AD fall below a predetermined threshold value. Advantageously, a gradual approximation of the centering to a predetermined accuracy value takes place, reaching the criterion for the accuracy to be achieved representing the termination condition.

In another embodiment of the method according to the invention, the deviations are determined on the basis of a phantom which is positioned in an examination area of the X-ray imaging system. Advantageously, the determination of the deviations and the calibration can take place via imaging of the phantom, the phantom being mapped on the detector surface of the X-ray detector. Provided that the phantom is correctly positioned, the pose of the emitter and the X-ray detector therefore does not have to be measured from the outside but can be determined by the recording of a test image.

The phantom preferably comprises a spiral arrangement of spheres, which are mapped onto the detector surface of the X-ray detector. By mapping the spiral arrangement of spheres onto the detector surface of the X-ray detector, the position and orientation of the two components relative to the phantom can be measured particularly easily and precisely via mapping.

Alternatively, the pose of the emitter and the X-ray detector can also be determined by measuring their pose from the outside. In this variant, the use of a phantom can be omitted.

Particularly preferably, the method according to one or more example embodiments is applied to a plurality of predetermined poses of the emitter and/or the X-ray detector, which form a trajectory. In particular, if a three-dimensional image of an examination area is to be generated, the examination area must be visually captured from different angles, preferably from an angular range of at least 200 degrees. The emitter and/or the X-ray detector can also be assigned a trajectory which they must follow in each case in order to visually capture the examination area from different directions. Advantageously, not just one position of the emitter and the X-ray detector is approached during the adjustment, but a plurality of positions or poses, which are located on a trajectory which is used or defined to record projection images of an examination area from different directions and to combine the projection images into a three-dimensional image. The 3D image is preferably generated by a filtered back projection of the projection images. The projection images are recorded from a plurality of predetermined positions.

It is highly preferable that the determined trajectory is smoothed by a compensation curve. Advantageously, more uniform imaging can be achieved from the different angles. The adjustment or centering described above refers to a systematic, reproducible deviation. However, the system can also display non-reproducible deviations which are caused, for example, by small vibrations. For this reason, it is advantageous if such a trajectory with random deviations is smoothed somewhat more. Alternatively, the deviations can also be measured several times and averaged to suppress this non-reproducible part before the actual correction.

In a preferred embodiment of the method according to the invention, the trajectory is determined by measuring the actual poses of the emitter and/or the X-ray detector several times and by averaging the measured values several times. Advantageously, the non-reproducible deviations described above are reduced.

In addition to an adjustment before imaging, which is preferably carried out once after the installation of an X-ray imaging system or at predetermined intervals, for example once a year, a calibration of a trajectory can also be carried out after image acquisition. Such a calibration makes it possible to take into account minimal deviations in the actual positions and orientations of the emitter and the X-ray detector during image reconstruction. Calibration is preferably carried out with each imaging or at a predetermined time interval or usage interval in order to make the image reconstruction precise.

The calibration of the trajectory corresponds to the step for determining the actual pose. This determined pose can be used directly for image reconstruction or just to correct the trajectory.

A phantom suitable for calibration, the “PDS-2” phantom, is described in “Improving 3D Image Quality of X-ray C-Arm Imaging Systems by using Properly Designed Pose Determination Systems for Calibrating the Projection Geometry” by Norbert Strobel et al., SPIE Medical Imaging 2003.

Depending on the manufacturer, there may also be different-looking phantoms for this step.

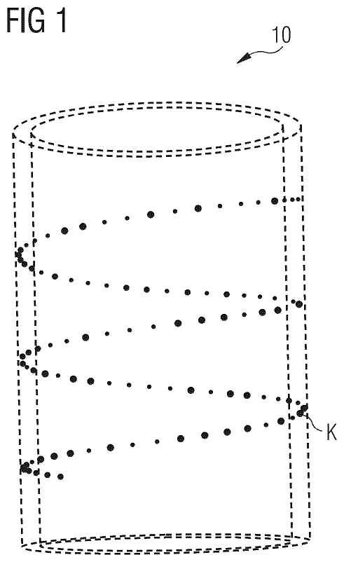

FIG. 1 shows a diagrammatic view of a phantom 10a with a number of opaque spheres K which are arranged in a spiral. The pattern created by the opaque spheres on the detector surface of an X-ray detector or the resulting projection image can be used to determine a pose of an emitter and an X-ray detector.

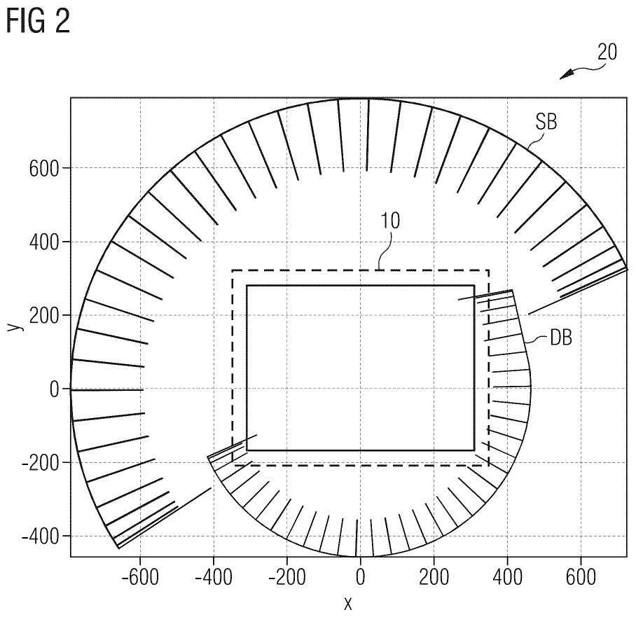

FIG. 2 shows a diagrammatic top view of the trajectory SB of an emitter and the trajectory DB of an X-ray detector of an X-ray imaging system 20. An image area 10, in which a patient can be positioned and mapped, is shown in the inner area between the two trajectories SB, DB.

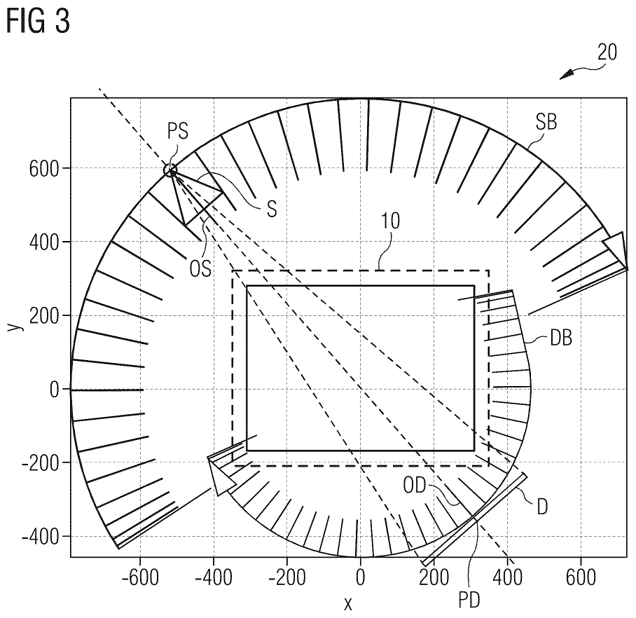

FIG. 3 illustrates a top view of an X-ray imaging system 20 with an emitter S and an X-ray detector D and the trajectories SB, DB already shown in FIG. 2 as well as the beam path (drawn as a broken line between the emitter S and the X-ray detector D) of the X-rays emitted by the emitter S. In FIG. 3 the emitter S and the X-ray detector D have an ideal opposite position PS, PD and an ideal orientation OS, OD rotated by 180°. In this ideal arrangement, the X-ray beam is projected in a centered manner by the emitter S onto the X-ray detector D.

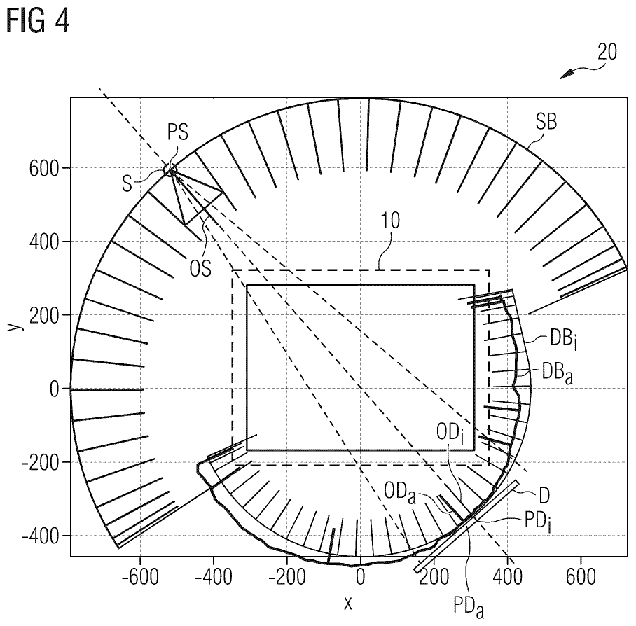

FIG. 4 shows a top view of the X-ray imaging system 20 already illustrated in FIG. 3, the actual trajectory DBa of the X-ray detector D being slightly shifted and rotated with respect to an ideal trajectory DBi or predetermined trajectory of the X-ray detector D. As indicated in FIG. 4, the X-ray beam of the emitter S no longer strikes the X-ray detector D centrally now, so that on one side (bottom left) of the detector surface of the X-ray detector D an edge is not irradiated and, on the side opposite this side (top right) of the detector surface of the X-ray detector D the irradiation goes beyond the edge of the detector surface of the X-ray detector D.

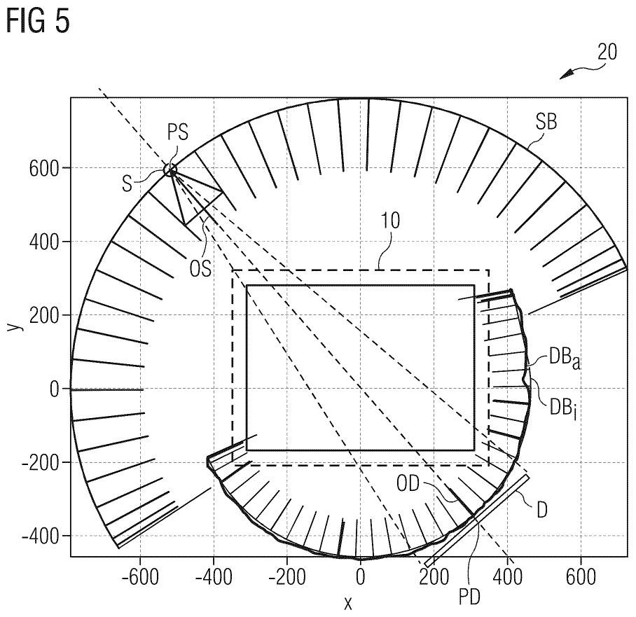

FIG. 5 shows a top view of the X-ray imaging system 20 already shown in FIG. 3 and FIG. 4, the trajectory DBa of the X-ray detector D having been corrected. The corrected actual trajectory DBa of the X-ray detector D now lies almost exactly on the original ideal trajectory DBi of the X-ray detector D. A further adjustment would be possible, for example, by smoothing the corrected actual trajectory DBa of the X-ray detector D, for example a compensation curve being laid through the corrected actual trajectory DBa of the X-ray detector D and a correction of the predetermined trajectory being carried out accordingly.

The purpose of a smoothing operation is to remove the non-reproducible portion from the actual trajectory, i.e. the actual trajectory DBa. This requires the measurement of the entire trajectory. The 3-dimensional positions and orientations can, for example, be smoothed by a convolution with a Gaussian kernel (comparable to a noise reduction operation). In other words, the smoothed actual positions are obtained from the actual positions and are then used instead of the actual positions to calculate the correction values.

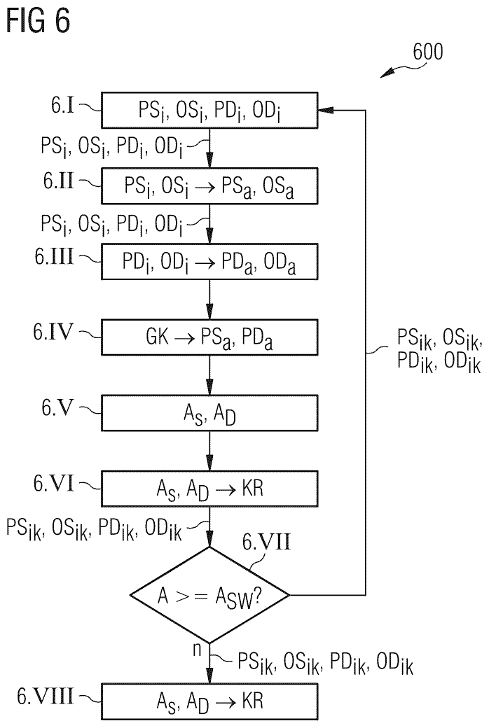

FIG. 6 shows a flow chart 600 which illustrates the method for centering an X-ray imaging system 20 (see FIG. 2 to FIG. 5) with an emitter with a variable pose and an X-ray detector with a variable pose which can be controlled independently of the variable pose of the emitter according to an exemplary embodiment of the invention.

In step 6.I, the position values of the emitter S and the X-ray detector D are set for different “axes”, which correspond to different orientation values. I.e. predetermined ideal values for different positions PSi, PDi and orientations OSi, ODi are transferred to the control of the emitter S and the X-ray detector D.

In step 6.II, a first position PSi and a first axis or orientation OSi of the emitter S is controlled, an actual position PSa and an actual orientation OSa being achieved by the emitter S, however. The more or less pronounced deviation between the ideal position PSi and the actual position PSa and/or the ideal orientation OSi and the actual orientation OSa leads to a decentering of the X-ray beam on the detector surface of the X-ray detector D of the X-ray imaging system 20.

In step 6.III, a first position PDi and a first axis or orientation ODi of the X-ray detector D is controlled, an actual position PDa and an actual orientation ODa being achieved by the X-ray detector, however. The more or less pronounced deviation between the ideal position PDi and the actual position PDa results in a decentering of the X-ray beam on the detector surface of the X-ray detector D of the X-ray imaging system 20.

In step 6.IV, a geometry calibration GK is performed, a phantom 10 positioned in an examination area being mapped on the detector surface of the X-ray detector D. During the geometry calibration, the actual positions PSa, PDa of the emitter S and the X-ray detector D of the X-ray imaging system 20 are determined. Likewise, the actual orientations OSa, ODa of the emitter S and the X-ray detector D are measured or determined.

In step 6.V, deviations AS, AD between predetermined ideal poses of the emitter S and the X-ray detector D are determined on the basis of the known ideal values and the actual values of the positions and orientations of the emitter S and the X-ray detector D measured in step 6.IV.

In step 6.VI, a correction KR takes place of the values of the ideal position PDi and the ideal orientation ODi of the X-ray detector D as well as a correction of the values of the ideal position PSi of the emitter S and the ideal orientation oSi of the emitter S, correction values PSik, OSik, PDik, ODik being generated.

In step 6.VII, the adjustment carried out in step 6.VI is checked via a renewed geometry calibration, i.e. in particular an image recording of the phantom 10 and an inspection, the actual poses of the emitter S and the X-ray detector D being compared with the values PSi, OSi, PDi, ODi set in step 6.I. If it is determined during step 6.VII that a predetermined tolerance or deviation ASW is not exceeded by the determined deviation A, which in FIG. 6 is characterized by “n”, step 6.VIII is commenced, in which the X-ray imaging system 20 is enabled for operation with a corresponding correction KR by using the correction values PSik, OSik, PDik, ODik for controlling the emitter S and the X-ray detector D. If it is determined in step 6.VII that the predetermined tolerance or deviation is still exceeded, step 6.I is commenced and the adjustment is carried out again, the correction values PSik, OSik, PDik, ODik now being used for control in steps 6.II and 6.III.

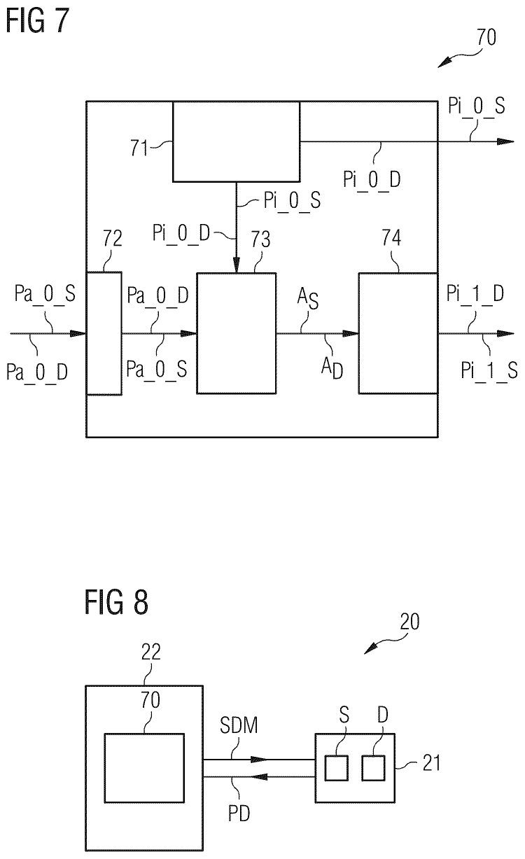

FIG. 7 shows a diagrammatic view of a centering facility 70 according to an exemplary embodiment of the invention.

The centering facility 70 has a control unit 71 for controlling a predetermined pose Pi_0_S (which comprises a first position PSi and a first axis or orientation OSi of an emitter S) with an emitter S of an X-ray imaging system and for controlling a pose Pi_0_D (which comprises a first position PDi and a first axis or orientation ODi of an X-ray detector) of the X-ray detector D corresponding to the predetermined pose Pi_0_S of the emitter S.

Part of the centering facility 70 is also a measuring unit 72 for determining the actual pose Pa_0_S (which comprises an actual position PSa of the emitter S of the X-ray imaging system 20 and an actual orientation OSa of the emitter S of the X-ray imaging system 20) of the emitter S and for determining the actual pose Pa_0_D (which comprises an actual position PDa of the X-ray detector D of the X-ray imaging system 20 and an actual orientation ODa of the X-ray detector D) of the X-ray detector D.

The centering facility 70 also comprises a deviation determination unit 73 for determining a first deviation AS of the actual pose Pa_0_S of the emitter from the predetermined pose Pi_0_S of the emitter S and for determining a second deviation AD of the actual pose Pa_0_D of the X-ray detector from the predetermined pose Pi_0_D of the X-ray detector D.

Furthermore, the centering facility 70 has a correction determination unit 74 for determining a corrected predetermined pose Pi_1_S of the emitter S based on the first deviation AS and for determining a corrected predetermined pose Pi_1_D of the X-ray detector D based on the second deviation AD. The correction values can also be transmitted to the control unit 71 in order to control the emitter S and the X-ray detector D with the corrected values and to adjust the poses of the emitter S and the X-ray detector D.

FIG. 8 shows a diagrammatic view of an X-ray imaging system 20 according to an exemplary embodiment of the invention. The X-ray imaging system 20 comprises the centering facility 70 shown in FIG. 7 as well as a scan unit 21 with an emitter S and an X-ray detector D. The centering facility 70 is part of a control facility 22 which is configured to control the scan unit 21 during imaging. Modified control data SDM is transmitted from the control facility 22 to the scan unit 21. The control data SDM is modified by the centering facility 70 in the manner illustrated in FIG. 1 to FIG. 7 in order to arrange the X-ray detector D and the emitter S of the scan unit 21 in the correct positions and align them with a correct orientation during imaging. Furthermore, projection data from a phantom (not shown) is received from the X-ray detector D by the control facility 22 in order to determine therefrom the input data of the centering facility 70 shown in FIG. 7, i.e. the actual pose Pa_0_S of the emitter S and the actual pose Pa_0_D of the X-ray detector D and to transmit this data to the centering facility 70 (see also FIG. 7).

Finally, it is pointed out again that the methods and apparatuses described above are merely preferred exemplary embodiments of the invention and that the invention can be varied by a person skilled in the art without departing from the scope of the invention, insofar as it is defined by the claims. For the sake of completeness, it is also pointed out that the use of the indefinite articles “a” or “an” does not exclude the possibility of the features concerned also being present several times. Likewise, the term “unit” does not exclude the possibility that it may consist of several components, which may also be spatially distributed. Regardless of the grammatical gender of a particular term, persons with male, female or other gender identities are included.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing facility) or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for centering an X-ray imaging system with an emitter with a variable pose and an X-ray detector with a variable pose, the variable pose of the X-ray detector being independently controllable from the variable pose of the emitter, the method comprising:
i) controlling a predetermined pose of the emitter;
ii) controlling a predetermined pose of the X-ray detector corresponding to the predetermined pose of the emitter;
iii) determining an actual pose of the emitter;
iv) determining an actual pose of the X-ray detector;
v) determining a first deviation of the actual pose of the emitter from the predetermined pose of the emitter;
vi) determining a second deviation of the actual pose of the X-ray detector from the predetermined pose of the X-ray detector;
vii) determining a corrected predetermined pose for centering the emitter based on the first deviation; and
viii) determining a corrected predetermined pose for centering the X-ray detector based on the second deviation.

2. The method of claim 1, further comprising:
repeating the steps i) to viii) if at least one of the first deviation or the second deviation exceed a predetermined threshold value, wherein the steps i) and ii) include using preceding corrected predetermined poses as predetermined poses, and using original predetermined poses in steps vi) and vii) for determining the first deviation and the second deviation.

3. The method of claim 1, further comprising:
repeating the steps i) to viii) iteratively until at least one of the first deviation or the second deviation is below a predetermined threshold value.

4. The method of claim 1, wherein the determining the actual pose of the emitter, the determining the actual pose of the X-ray detector, the determining the first deviation, and the determining the second deviation are based on a phantom positioned in an examination area of the X-ray imaging system.

5. The method of claim 4, wherein the phantom comprises a spiral arrangement of spheres mapped on the X-ray detector.

6. The method of claim 5, further comprising:
performing steps i) to viii) to a plurality of predetermined poses of at least one of the emitter or the X-ray detector to determine a trajectory.

7. The method of claim 6, wherein the determined trajectory is smoothed by a compensation curve.

8. The method of claim 6, wherein the trajectory is determined by multiple measurements of at least one of the actual poses of the emitter or the actual poses of the X-ray detector and by averaging the multiple measurements.

9. The method of claim 1, wherein further comprising:
centering the X-ray imaging system by controlling the emitter with the corrected predetermined pose of the emitter and controlling the X-ray detector with the corrected predetermined pose of the X-ray detector.

10. The method of claim 9, wherein the centering centers the X-ray imaging system such that a center beam of the emitter strikes a center of the X-ray detector and the center beam strikes a detector surface of the X-ray detector vertically.

11. A centering facility, comprising:
a control unit configured to control a predetermined pose of an emitter and configured to control a corresponding predetermined pose of an X-ray detector, the control unit configured to control the predetermined pose of the X-ray detector independently of the predetermined pose of the emitter;
a measuring unit configured to determine an actual pose of the emitter and determine an actual pose of the X-ray detector;
a deviation determination unit configured to determine a first deviation of the actual pose of the emitter from the predetermined pose of the emitter and for determining a second deviation of the actual pose of the X-ray detector from the predetermined pose of the X-ray detector; and
a correction determination unit configured to determine a corrected predetermined pose for centering the emitter based on the first deviation and determine a corrected predetermined pose for centering the X-ray detector based on the second deviation.

12. A centering facility of claim 11, wherein the control unit is configured to control the emitter with the corrected predetermined pose of the emitter and to control the X-ray detector with the corrected predetermined pose of the X-ray detector.

13. An X-ray imaging system, comprising:
an emitter with variable pose control;
an X-ray detector the pose of which can be variably controlled independently of the pose of the emitter; and
the centering facility of claim 11.

14. A non-transitory computer program product, comprising commands which, when executed by a computer, cause the computer to perform the method of claim 1.

15. A non-transitory computer-readable storage medium comprising commands which, when executed by a computer, cause the computer to perform the method of claim 1.

16. The method of claim 2, further comprising:
repeating the steps i) to viii) iteratively until at least one of the first deviation or the second deviation is below a predetermined threshold value.

17. The method of claim 16, wherein the determining the actual pose of the emitter, the determining the actual pose of the X-ray detector, the determining the first deviation, and the determining the second deviation are based on a phantom positioned in an examination area of the X-ray imaging system.

18. The method of claim 7, wherein the trajectory is determined by multiple measurements of at least one of the actual poses of the emitter or the actual poses of the X-ray detector and by averaging the multiple measurements.

19. The method of claim 18, wherein further comprising:
centering the X-ray imaging system by controlling the emitter with the corrected predetermined pose of the emitter and controlling the X-ray detector with the corrected predetermined pose of the X-ray detector.

20. The method of claim 19, wherein the centering centers the X-ray imaging system such that a center beam of the emitter strikes a center of the X-ray detector and the center beam strikes a detector surface of the X-ray detector vertically.
